(19) United States
(12) Patent Application Publication
(10) Pub. No.: US 2009/0286744 A1
(43) Pub. Date: Nov. 19, 2009

(54) Treatment of asthma

(75) Inventor: Mark L. Witten, Tucson, AZ (US)

Correspondence Address: BANNER & WITCOFF, LTD., 1100 13th STREET, N.W., SUITE 1200, WASHINGTON, DC, 20005-4051, US

(73) Assignee: IMMUNEREGEN BIOSCINECES INC., SCOTTSDAL ARIZONA (US)

(21) Appl. No.: 11/887,105

(22) PCT Filed: Mar. 31, 2006
(86) PCT No.: PCT/US2006/011833

§ 371 Date: Dec. 31, 2008

Related U.S. Application Data
(60) Provisional application No. 60/667,062, filed on Apr. 1, 2005.

(51) Int. Cl. A61K 38/08; A61P 11/06

(52) U.S. Cl. 514/15

(57) ABSTRACT

Substance P or its analogs are useful for treating asthma. The active agents can be administered orally, intravenously, intramuscularly, intranasally, topically. Disease severity is reduced by substance P treatment.

### BACKGROUND OF THE INVENTION

Asthma is a chronic respiratory disease characterized by inflammation of the airways; hypersensitivity of the airways, and often an obstruction of airflow. Airflow obstruction may be due to bronchospasms and/or mucous secretion. Many factors can trigger an asthma attack, including allergens, infections, exercise, abrupt changes in the weather, or exposure to airway irritants, such as tobacco smoke and airborne pollutants.

There is a continuing need in the art for means of treating asthma.

### SUMMARY OF THE INVENTION

According to one embodiment of the invention a method is provided for treating asthma in a patient. An agent is administered to a human with asthma. The agent is selected from the group consisting of: substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (0₂) 11-Substance P, and [p-Cl-Phe^{7,8}]-substance P. The treatment results in a reduction in the severity of asthma symptoms.

### BRIEF DESCRIPTION OF THE DRAWINGS

FIG. 1 shows SP-induced degradation of PARP-1 expression in alveolar macrophages.

### DETAILED DESCRIPTION OF THE INVENTION

It is a discovery of the present inventor that Substance P and its bioactive analogs, such as Sar⁹, Met (0₂) 11-Substance P, are beneficial for treating asthma. Substance P and its analogs can be used to reduce the severity of asthma symptoms. Substance P and its analogs also inhibit poly(ADP-ribose) polymerase-1 (PARP), an enzyme implicated in the activation of airway inflammation.

Symptoms of asthma include wheezing, coughing, shortness of breath, tightness in the chest, reduced peak air flow numbers. Symptoms of severe asthma include severe coughing, wheezing, shortness of breath or tightness in the chest, difficulty talking or concentrating, shortness of breath upon walking, shallow and fast breathing or slower than usual breathing, hunched shoulders, nasal flaring, retractions, cyanosis, and peak airway flow numbers below 50% of personal best.

Substance P (RPKPQQFFGLM-NH₂; SEQ ID NO: 1) or a bioactive analog thereof such as Sar⁹, Met (0₂) 11-Substance P can be administered to treat asthma. The bioactive analog can be selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (0₂) 11-Substance P, and [p-Cl-Phe^{7,8}]-substance P. Other compounds which function in the same way can be identified by their ability to compete with substance P for binding to its receptor (NK-1) or for their ability to agonize the NK-1 receptor. Routine assays for such activities are known in the art and can be used.

The substance P or analog is administered by any route known in the art. These include topical, intramuscular, intravenous, subcutaneous, via aerosol, by instillation to the lungs, oral, and intranasal. Typically it is admixed with an appropriate vehicle, such as an aqueous liquid or a solid. Typical concentration ranges of substance P or its bioactive analog in the vehicle is between 0.001 and 10 μM, between 0.05 and 5 μM, or between 0.1 and 1 μM.

In one embodiment of the invention, the agent is administered in a timed-release formulation. The period of release may be, for example, from 12 to 24 hours.

Bioactive analogs, according to the invention are those which act as competitive inhibitors of SP by binding to the SP receptor (NK-1 receptor). The analogs may be agonists of the NK-1 receptor. Other derivatives as are known in the art and commercially available (e.g., from Sigma, St. Louis, Mo., or Polypeptide Laboratories A/S, Hillerod, Denmark) can be used. In addition, substance P fragments and derivatized substance P fragments may also be used. Substitution, deletion, or insertion of one to eight amino acid residues, and preferably from one to three amino acid residues, will lead to analogs which can be routinely tested for biological activity. In addition, functional groups may be modified on SP while retaining the same peptide backbone. Again, routine testing will determine which of such modifications do not adversely affect biological activity.

Suitable treatment regimens for treatment according to the present invention include one-time, monthly, weekly, daily or multiple daily treatments by topical application. Frequency may depend on the severity of symptoms or the exposure to an aggravating substance or condition. Suitable formulations of substance P for administration are any which are pharmaceutically or cosmetically acceptable and in which the substance P or bioactive analog retains its biological activity. Generally, such formulations include substance P dissolved in normal saline or other aqueous medium, in creams, in lotions, in ointments, or in gels. Other formulations for changing absorption and half-life characteristics can be used, including liposomal formulations and slow-release formulations.

Disease symptoms are improved by the present treatments. Reduction in symptoms may by complete upon extremely successful treatment. Reduction in the symptoms of at least 10%, 15%, 20%, 25%, 30%, 35%, 40%, or 50% are desirable. Even greater decreases are preferred.

### EXAMPLE 1

The mice were divided into cigar or cigarette smoking groups and administered one hour smoke exposure for five days/week for three consecutive weeks. Of course, it is well known that cigar smoke is very pungent/toxic even compared to cigarette smoke. Even habitual cigar smokers do not inhale the cigar smoke deeply into their lungs. Our C57BL/6 mice did not have the choice of inhaling the cigar smoke deeply within their lungs while they were in our IN-TOX exposure chamber.

We established a baseline pulmonary function value, administered 0.1 ml bolus of a 1 mg/ml dose of histamine via the mouse's endotracheal tube, waited two minutes, and then repeated the pulmonary function tests. The results with cigar/cigarette smoke demonstrated that Homspera can attenuate histamine-induced airway hyperreactivity. The data are the following—

Dynamic Lung Compliance is a measure of chronic obstructive pulmonary disease and increases in pulmonary emphysema. Inspiratory dynamic lung compliance is a sensitive indicator of airway hyperreactivity because asthma is a restrictive lung disease, obviously harder to get air into the lungs than out of the lungs (obstructive lung disease).

TABLE 1
Percent Increase in Inspiratory Dynamic Lung Compliance
Between Baseline and Post-Histamine Administration

| | |
|---|---|
| Controls (N = 7) | 317.0% increase |
| Cigar (N = 4) | 161.4% increase |
| Cigar + Homspera (N = 4) | 38.0% increase |
| Cigarette (N = 4) | 374.5% increase |
| Cigarette + Homspera (N = 4) | 226.4% increase |

These data demonstrate that in a cigar or cigarette smoke exposure-induced airway hyperreactivity model, Homspera treatment at 1 microMolar concentration can attenuate the development of airway hyperreactivity after histamine administration.

### EXAMPLE 2

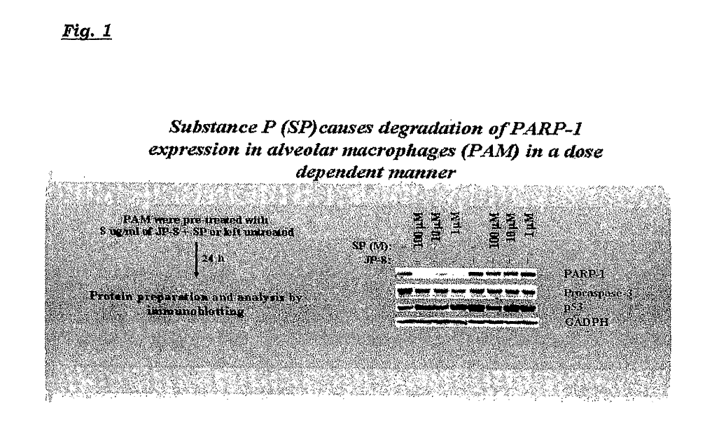

Alveolar macrophages (PAM) were pre-treated with 8 ug/ml of JP-8 and substance P, or left untreated. After 24 hours, protein was extracted from the PAM and analyzed by immunoblotting for PARP-1, procaspase-3, p53, and GAPDH. See FIG. 1. Substance P causes degradation of PARP-1 expression in alveolar macrophages in a dose-dependent manner.

I claim:

1. A method of treating an asthma patient, comprising:
administering to an asthma patient, an agent selected from the group consisting of: substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (0₂) 11-Substance P, and [p-Cl-Phe^{7,8}]-substance P, whereby severity of symptoms of asthma in the patient is decreased.

2. The method of claim 1 whereby inflammation in the lung is reduced.

3. The method of claim 1 whereby inflammation in the airways is reduced.

4. The method of claim 1 whereby poly(ADP-ribose) polymerase (PARP) activity in lung cells is inhibited.

5. The method of claim 1 wherein poly(ADP-ribose) polymerase (PARP) expression in lung cells is inhibited.

6. The method of claim 1 wherein the agent is administered as an aerosol to the airways.

7. The method of claim 1 wherein the agent is administered orally.

8. The method of claim 7 wherein the agent is administered in a time-release formulation.

9. The method of claim 8 wherein the agent is released over a period of between 12 and 24 hours.

10. The method of claim 1 wherein the agent is administered intravenously.

11. The method of claim 1 wherein the agent is administered intramuscularly.

12. The method of claim 1 wherein the agent is administered topically.

13. The method of claim 1 wherein Sar⁹, Met (0₂) 11-Substance P is administered.

14. The method of claim 1 wherein Substance P is administered.

15. The method of claim 1 wherein [Met-OH¹¹]-substance P is administered.

16. The method of claim 1 wherein [Met-OMe¹¹]-substance P is administered.

17. The method of claim 1 wherein [Nle¹¹]-substance P is administered.

18. The method of claim 1 wherein [Pro⁹]-substance P is administered.

19. The method of claim 1 wherein [Sar⁹]-substance P is administered.

20. The method of claim 1 wherein [Tyr⁸]-substance P is administered.


SEQUENCE LISTING (1 sequence)

SEQ ID NO: 1 - protein, 11 aa, Homo sapiens
RPKPQQFFGLM